(19) United States
(12) Patent Application Publication
(10) Pub. No.: US 2026/0199700 A1
(43) Pub. Date: Jul. 16, 2026

(54) Phototherapy Device

(71) Applicant: Dongguan Laiguang Electronic Technology Co., Ltd., Dongguan (CN)

(72) Inventor: Zekai LUO, Dongguan (CN)

(21) Appl. No.: 19/045,088

(22) Filed: Feb. 4, 2025

(30) Foreign Application Priority Data
Jan. 13, 2025 (CN) 2025200746665

(51) Int. Cl. A61N 5/06; A61N 5/00

(52) U.S. Cl. CPC A61N 5/0613; A61N 2005/005; A61N 2005/0642; A61N 2005/0645; A61N 2005/0654

(57) ABSTRACT

A phototherapy device includes a heat-conducting housing and a phototherapy module, a heat-conducting housing is capable of curved surface deformation under an external force; the heat-conducting housing has a curved surface state or a planar state, and the heat-conducting housing is further provided with a light-transmitting portion; a phototherapy module is provided in the heat-conducting housing; the phototherapy module is provided with a light output portion, and the light output portion is arranged corresponding to the light-transmitting portion for outputting light, which penetrates the light-transmitting portion and is emitted outwardly; and the phototherapy module exchanges heat with the heat-conducting housing, and the phototherapy module generates heat when outputting light, and the heat is dissipated outwardly via the heat-conducting housing.

### CROSS-REFERENCE TO RELATED APPLICATIONS

The present application claims priority to Chinese Patent Application No. 202520074666.5, filed on Jan. 13, 2025, the entire disclosure of which is incorporated herein by reference.

### TECHNICAL FIELD

The present application relates to the technical field of phototherapy devices, in particular to a phototherapy device.

### BACKGROUND

With the development of science and technology, a phototherapy device utilizes light output from an artificial light source or a natural light source for phototherapy to achieve a phototherapy effect on a user. In the related art, the existing phototherapy device includes a housing and a phototherapy module, the phototherapy module is connected to the housing, and the light output from the phototherapy module penetrates the light transmission portion and is ejected outwardly, so as to facilitate irradiation of the light output from the phototherapy module to the user, but the phototherapy module generates heat during the working process, and the heat can not be discharged outwardly, which results in a poorer heat dissipation effect of the existing phototherapy device.

### SUMMARY

The purpose of the present application is to provide a phototherapy device that makes full use of the heat-conducting effect of the heat-conducting housing, the phototherapy module generates heat when outputting light, and the heat is dissipated outwardly via the heat-conducting housing, and the heat-conducting housing improves the area of heat dissipation and improves the heat dissipation effect of the phototherapy device.

In order to realize the above purpose, the present application provides the following technical solution:
a phototherapy device, including:
a heat-conducting housing, capable of curved surface deformation under an external force; wherein the heat-conducting housing has a curved surface state or a planar state, and the heat-conducting housing is further provided with a light-transmitting portion;
a phototherapy module, provided in the heat-conducting housing;
the phototherapy module is provided with a light output portion, and the light output portion is arranged corresponding to the light-transmitting portion for outputting light, which penetrates the light-transmitting portion and is emitted outwardly; and
the phototherapy module exchanges heat with the heat-conducting housing, and the phototherapy module generates heat when outputting light, and the heat is dissipated outwardly via the heat-conducting housing.

In order to realize the above purpose, the present application provides the following technical solution: a phototherapy device, including:
a heat-conducting housing, being a heat-conducting member, wherein the heat-conducting housing is further provided with a light-transmitting portion;
a phototherapy module, provided in the heat-conducting housing;
the phototherapy module is provided with a light output portion, and the light output portion is arranged corresponding to the light-transmitting portion for outputting light, which penetrates the light-transmitting portion and is emitted outwardly; and
the phototherapy module exchanges heat with the heat-conducting housing, and the phototherapy module generates heat when outputting light, and the heat is dissipated outwardly via the heat-conducting housing

The beneficial effects of the present application compared to the related art are:

The present application provides a phototherapy device, a heat-conducting housing is capable of curved surface deformation under an external force; the heat-conducting housing has a curved surface state or a planar state, and the heat-conducting housing is further provided with a light-transmitting portion; a phototherapy module is provided in the heat-conducting housing; the phototherapy module is provided with a light output portion, and the light output portion is arranged corresponding to the light-transmitting portion for outputting light, which penetrates the light-transmitting portion and is emitted outwardly; and the phototherapy module exchanges heat with the heat-conducting housing, and the phototherapy module generates heat when outputting light, and the heat is dissipated outwardly via the heat-conducting housing. At this time, it makes full use of the heat-conducting effect of the heat-conducting housing, the phototherapy module generates heat when outputting light, and the heat is dissipated outwardly via the heat-conducting housing, and the heat-conducting housing improves the area of heat dissipation and improves the heat dissipation effect of the phototherapy device.

### BRIEF DESCRIPTION OF THE DRAWINGS

In order to more clearly illustrate the technical solutions in the embodiments of the present application, the accompanying drawings to be used in the description of the embodiments will be briefly described below. Obviously, the accompanying drawings in the following description are only some of the embodiments of the present application, and other accompanying drawings may be obtained from these drawings without creative labor for those skilled in the art.

For a more complete understanding of the present application and its beneficial effects, the following description will be given in conjunction with the accompanying drawings. The same accompanying drawing symbols in the following description indicate the same parts.

FIG. 1 is a schematic diagram of a phototherapy device of the present application.

FIG. 2 is an exploded view of the phototherapy device of the present application.

FIG. 3 is a schematic diagram of a heat-conducting housing of the phototherapy device of the present application.

FIG. 4 is a schematic diagram of a phototherapy module of the phototherapy device of the present application.

FIG. 5 is another schematic diagram of the phototherapy device.

FIG. 6 is a partially enlarged view at A in FIG. 5.

FIG. 7 is an exploded view of the phototherapy device.

FIG. 8 is a schematic view of the phototherapy device in a first winding band and a second winding band.

FIG. 9 is a partially enlarged view at B in FIG. 8.

FIG. 10 is a schematic view of a to-be-connected member of the phototherapy device.

FIG. 11 is an exploded view of the to-be-connected member of the phototherapy device.

FIG. 12 is an exploded view of another state of the to-be-connected member of the phototherapy device.

FIG. 13 is a partially enlarged view at C in FIG. 12.

FIG. 14 is a schematic view of a heat-conducting housing of the phototherapy device.

FIG. 15 is a schematic diagram of the to-be-connected member of the phototherapy device as a multi-angle holder.

FIG. 16 is a schematic diagram of the to-be-attached member of the phototherapy device as a floor stand.

FIG. 17 is a schematic diagram of the disassembly of an elastic snap of the phototherapy device.

FIG. 18 is a schematic diagram of the disassembly of a locking structure of the phototherapy device.

FIG. 19 is a schematic diagram of a thread-through structure of a first winding band of the phototherapy device.

FIG. 20 is another schematic diagram of the thread-through structure the first winding band of the phototherapy device.

### DETAILED DESCRIPTION OF THE EMBODIMENTS

The technical solutions in the embodiments of the present application will be clearly and completely described below in conjunction with the accompanying drawings in the embodiments of the present application. Obviously, the described embodiments are only a part of the embodiments of the present application, and not all of the embodiments. Based on the embodiments in the present application, all other embodiments obtained by those skilled in the art without making creative labor fall within the scope of the present application.

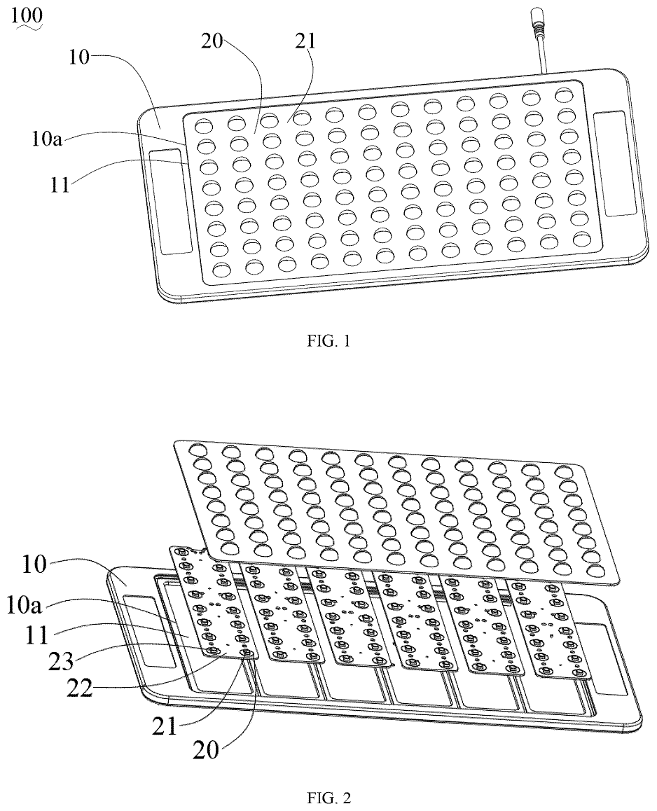

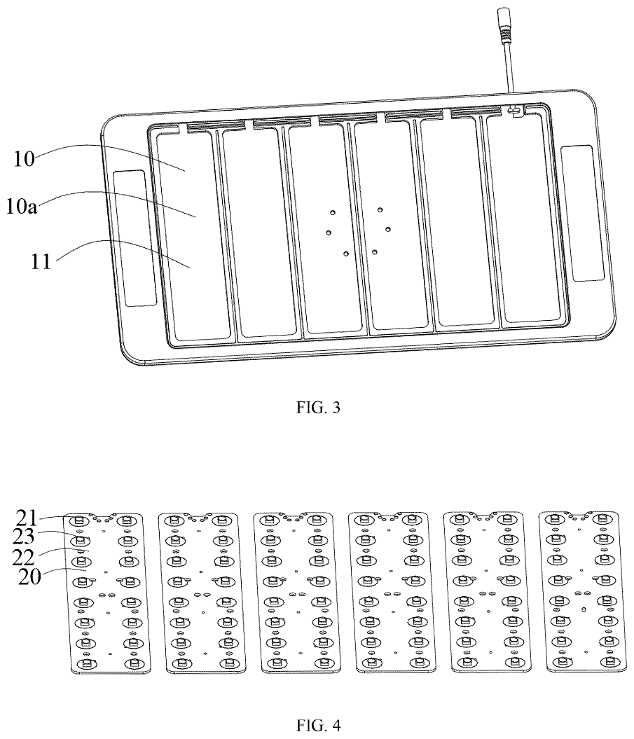

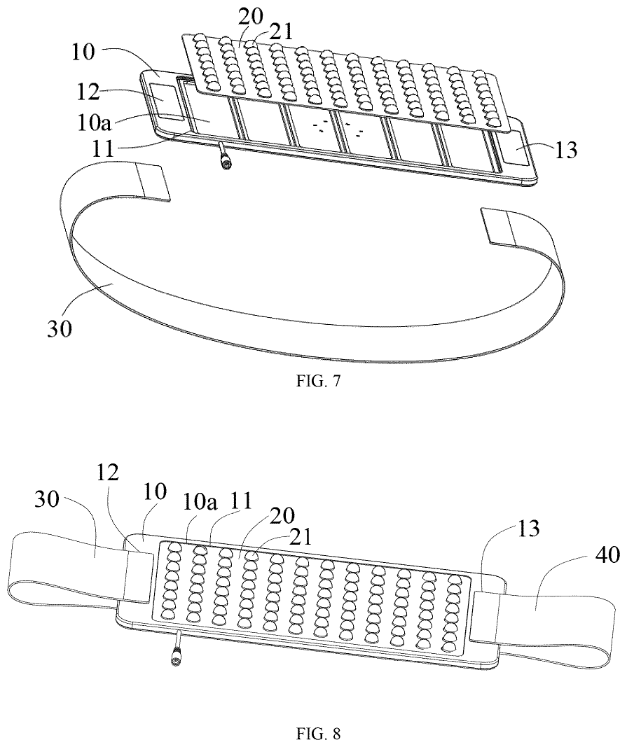

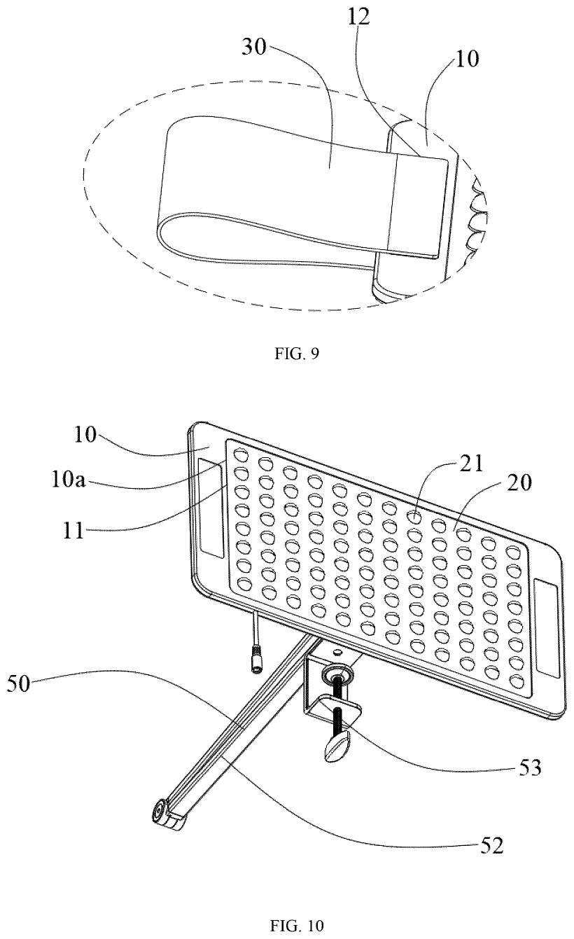

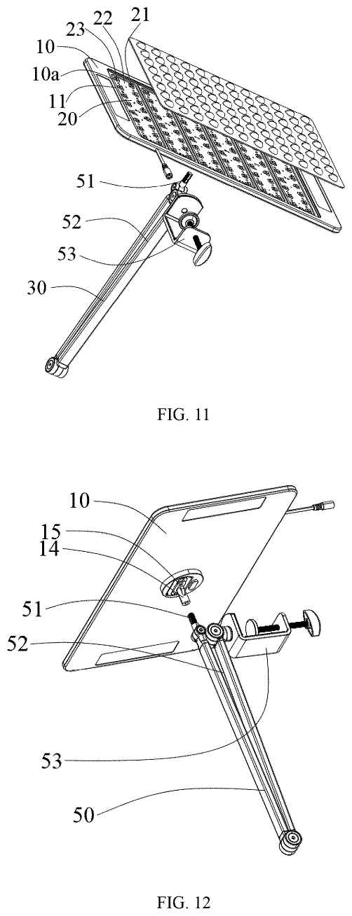

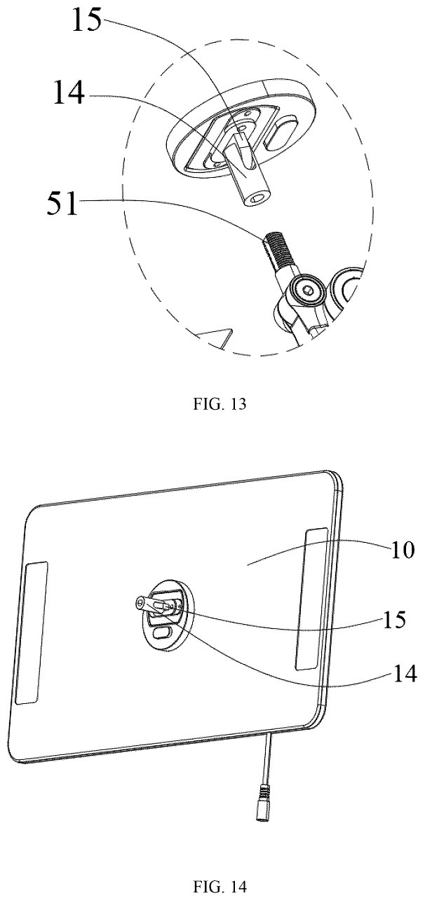

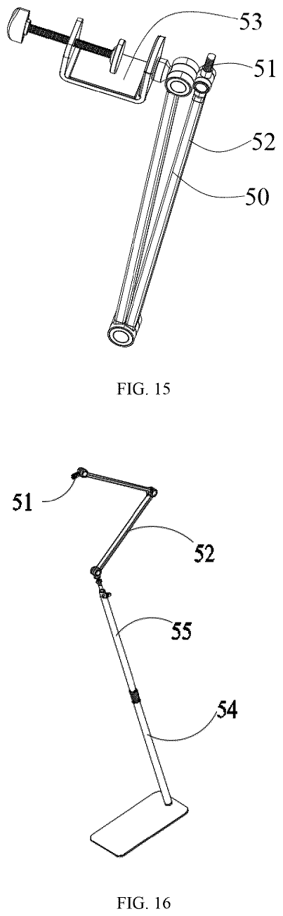

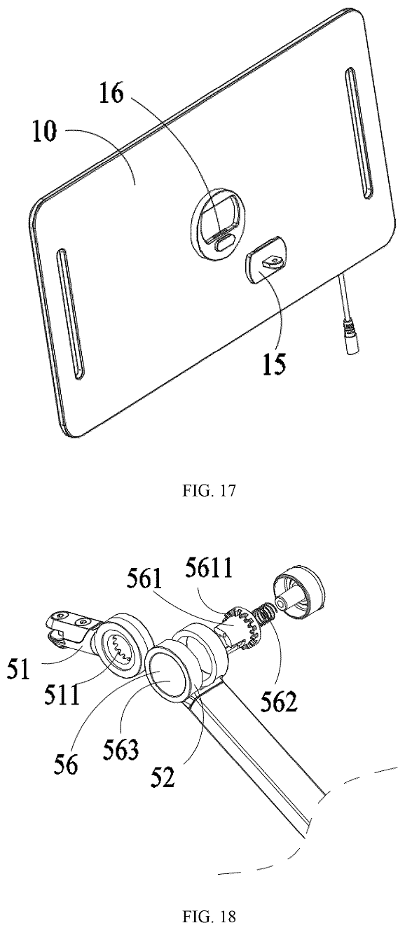

Referring to FIGS. 1 to 20, the present application provides a phototherapy device 100, the phototherapy device 100 is used to output light to a user. Optionally, the light is red light, blue light or infrared light, and the red light, blue light or infrared light has a phototherapy effect on the skin of the user.

Referring to FIG. 1~4, in the present application, the phototherapy device 100 includes a heat-conducting housing 10 and a phototherapy module 20, the phototherapy module 20 is provided on an inner side of the heat-conducting housing 10, and the heat-conducting housing 10 is capable of curved surface deformation under an external force. The heat-conducting housing 10 has a curved surface state or a planar state, and the heat-conducting housing 10 is also provided with a light-transmitting portion 11. The phototherapy module 20 is provided on the heat-conducting housing 10. The phototherapy module 20 is provided with a light output portion 21, the light output portion 21 is arranged corresponding to the light-transmitting 11 and is used to output light, which penetrates the light-transmitting 11 and is emitted outwardly. The phototherapy module 20 exchanges heat with the heat-conducting housing 10, and the phototherapy module 20 generates heat when outputting light, which is dissipated outwardly via the heat-conducting housing 10. At this time, it makes full use of the heat-conducting effect of the heat-conducting housing 10, the phototherapy module 20 generates heat when outputting light, and the heat is dissipated outwardly via the heat-conducting housing 10, and the heat-conducting housing 10 improves the area of heat dissipation and improves the heat dissipation effect of the phototherapy device 100.

Referring to FIG. 1~3, in the present application, the heat-conducting housing 10 serves as a support component of the phototherapy device 100, and the heat-conducting housing 10 is capable of curved surface deformation under the external force, in order to realize a variety of shape changes of the heat-conducting housing 10. The heat-conducting housing 10 has a curved surface state or a planar state, so that heat dissipation of the heat-conducting housing 10 in different states can improve the heat dissipation effect of the phototherapy device 100.

In addition, when the heat-conducting housing 10 is in the planar state, it is convenient for the heat-conducting housing 10 to be adapted to a planar phototherapy module 20, thereby facilitating the phototherapy module 20 to be in the planar state to output light toward the user, so as to realize that the phototherapy module 20 performs a planar phototherapy on the user, and when the heat-conducting housing 10 is in the curved surface state, it is convenient for the heat-conducting housing 10 to be adapted to the phototherapy module 20 of the curved surface, thereby facilitating the outer surface of the heat-conducting housing 10 to be in curved attachment with the waist or or arm of the user, which ensures that the heat-conducting housing 10 fits the waist or arm of the user. The heat-conducting housing 10 is also provided with a light-transmitting portion 11, which is on a front side of the heat-conducting housing 10, and the light-transmitting portion 11 is configured for the light to penetrate through.

Referring to FIG. 1~3, the heat-conducting housing 10 is provided with an accommodation slot 10a, the accommodation slot 10a serves as an internal space of the heat-conducting housing 10, the accommodation slot 10a is arranged corresponding to the light-transmitting portion 11. The phototherapy module 20 is accommodated within the accommodation slot 10a so as to allow the phototherapy module 20 to connect to the interior of the heat-conducting housing 10 through the accommodation slot 10a, the phototherapy module 20 is directly in contact with an inner side wall of the accommodation slot 10a and exchanges heat with the heat-conducting housing 10, so as to facilitate the heat generated by the phototherapy module 20 outputting light to be directly conducted to the heat-conducting housing 10, in order to transfer the heat of the phototherapy module 20 to the heat-conducting housing 10, so as to achieve heat dissipation effect of the phototherapy module 20.

Referring to FIG. 1~3, the heat-conducting housing 10 is a heat-conducting member to realize heat exchange between the heat-conducting housing 10 and the phototherapy module 20, so as to facilitate the heat-conducting housing 10 to absorb the heat generated by the phototherapy module 20 when outputting the light. The heat-conducting housing 10 is exposed to the external environment and dissipates the heat generated by the phototherapy module 20 outwardly in different directions, so as to facilitate the heat-conducting housing 10 to contact the airflow of the external environment, to further accelerating the heat dissipation effect of the heat-conducting housing 10. Optionally, the heat-conducting housing 10 is a silicone housing, a silicone plate, a heat-conducting metal plate or a heat-conducting sheet, and the silicone housing, the silicone plate, the heat-conducting metal plate or the heat-conducting sheet are capable of deformation under an external force and have a heat-conducting effect.

Referring to FIG. 1~2 and 4, in the present application, the phototherapy module 20 is provided on an inner side of the heat-conducting housing 10, and the phototherapy module 20 is provided on the heat-conducting housing 10, so as to facilitate the connection of the phototherapy module 20 to the inner side of the heat-conducting housing 10. The phototherapy module 20 is provided with a light output portion 21, the light output portion 21 is arranged corresponding to the light-transmitting portion 11 and is configured for outputting light, the light penetrates through the light-transmitting portion 11 and is emitted outwardly, so that the light output portion 21 outputs light, and the light passes through the light-transmitting portion 11 to irradiate to the external environment, to achieve the effect of phototherapy. The phototherapy module 20 exchanges heat with the heat-conducting housing 10, and the phototherapy module 20 generates heat when outputting light, and the heat dissipates outwardly via the heat-conducting housing 10, which fully utilizes the heat-conducting effect of the heat-conducting housing 10, and the phototherapy module 20 generates heat when outputting light, and the heat is dissipated outwardly via the heat-conducting housing 10, the heat-conducting housing 10 improves the area of heat dissipation and improves the heat dissipation effect of the phototherapy device 100.

Referring to FIG. 1~2 and 4, the phototherapy module 20 includes a circuit board 22 and a plurality of lamp beads 23. The circuit board 22 is connected to an inner side wall of the accommodation slot 10a so as to facilitate the circuit board 22 to be connected to the inner side of the heat-conducting housing 10, and the circuit board 22 is in direct contact with the inner side wall of the accommodation slot 10a so as to facilitate the heat exchange between the circuit board 22 and the inner side wall of the accommodation slot 10a, thereby facilitating that heated generated by the circuit board 22 during working is conducted to the heat-conducting housing 10, so as to transfer heat from the circuit board 22 to the heat-conducting housing 10, and thus the heat dissipation of the circuit board 22 is achieved.

Referring to FIG. 1~2 and 4, the plurality of light beads 23 are provided on a front side of the circuit board 22 toward the light-transmitting portion 11, the plurality of light beads 23 are electrically connected to the circuit board 22, so as to facilitate the connection of the plurality of light beads 23 to the circuit board 22, the plurality of light beads 23 output light to the light-transmitting portion 11 and to the external environment, so as to facilitate the plurality of light beads 23 outputting light toward a user under the state of energized power of the circuit board 22 to perform a phototherapy effect, thereby realizing phototherapy to the user. Optionally, the lamp beads 23 are led lamp beads, and the lamp beads 23 output red light, infrared light for phototherapy.

Referring to FIG. 1~2 and 4, a plurality of phototherapy modules 20 are spaced apart along a lengthwise direction of the heat-conducting housing 10 and extended along the widthwise direction of the heat-conducting housing 10, and the plurality of phototherapy modules 20 improve the area of phototherapy of the phototherapy module 20 corresponding to the user, thereby improving the phototherapy efficiency of the phototherapy module 20 corresponding to the user.

Optionally, an articulation member is provided between the plurality of the phototherapy modules 20, and two adjacent phototherapy modules are swung through the articulation member to realize angular adjustment, at which time the articulation member may be provided between two adjacent circuit boards 22, thereby realizing the deformation of the heat-conducting housing 10.

In addition, a side wall of the circuit board 22 back to the lamp bead 23 is connected to a base, and the articulation member may be set between two adjacent bases for achieving the angle adjustment, and thus the angle adjustment of the two adjacent the phototherapy modules is achieved, and thus the deformation of the heat-conducting housing 10 is achieved. Optionally, the base is a heat dissipation seat, and the articulation member is a hinge and other components.

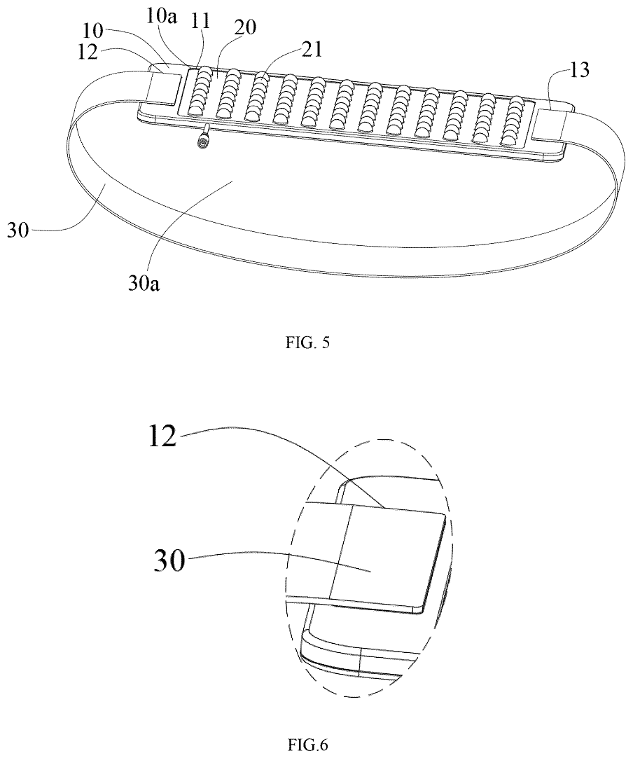

Referring to FIGS. 5 to 7, the heat-conducting housing 10 is provided with a first connection portion 12 and a second connection portion 13, and the first connection portion 12 and the second connection portion 13 are provided at different positions of the heat-conducting housing 10. The phototherapy device 100 further includes a first winding band 30, the first winding band 30 is connected to the heat-conducting housing 10, One end of the first winding band 30 is connected to the first connection portion 12, and another end of the first winding band 30 is directly or indirectly connected to the second connection portion 13, the first winding band 30 is provided with a winding space 30a, the winding space 30a is configured for a user's body to pass through, so as to facilitate increasing the first winding band 30 to wrap around the user's body, so as to facilitate the heat-conducting housing 10 to wrap around the user's arm or waist, avoiding the heat-conducting housing 10 from being unable to be connected corresponding to the user's body, and enriching the application scenarios of the phototherapy device 100.

Referring to FIG. 5~7, the first connection portion 12 and the second connection portion 13 are provided at the left end and the right end of the heat-conducting housing 10, respectively, and the first winding band 30 is provided between the first connection portion 12 and the second connection portion 13, and the first winding band 30 is provided in a horizontal direction. The first winding band 30 is connected to the first connection portion 12, and is connected to the second connection portion 13 along the left and right directions, so as to facilitate the first winding band 30 to be connected to the first connection part 12 and the second connection part 13 in the left and right directions, respectively, so as to realize that the first winding band 30 and the heat-conducting housing 10 form a winding space 30a. Optionally, the winding space 30a is ring-shaped.

Referring to FIG. 5~7, the first winding band 30 is an elastic first winding band 30 so that the first winding band 30 has an elastic effect, the first winding band 30 can be elastically stretched under the external force, so as to facilitate the change of the length of the first winding band 30, and the first winding band 30 elastically regulates the winding space 30a so as to facilitate the adjustment of the size of the winding space 30a, so as to facilitate the adaptability of the winding space 30a to a variety of different sizes of the user's body, to realize the two different sizes of the heat-conductive housing 10 can wrap around the user's arm or waist, such that the adaptability of the heat-conducting housing 10 is improved and the application scenarios of the phototherapy device 100 is enriched.

Referring to FIGS. 5 to 7, the connection structure provided between the first winding band 30 and the heat-conducting housing 10 is at least one of a snap connection structure, a locking connection structure, a velcro connection structure, a glued connection structure, and a thread-through structure. At this time, the first winding band 30 is connected to the first connection portion 12 or the second connection portion 13 by a snap connection, or the first winding band 30 is connected to the first connection portion 12 or the second connection portion 13 by a locking connection. The first winding band 30 is connected to the first connection part 12 or the second connection part 13 by a snap, or the first winding band 30 is connected to the first connection part 12 or the second connection part 13 by a locking method, or the first winding band 30 is connected to the first connection part 12 or the second connection part 13 by means of a velcro fastener, or the first winding band 30 is connected to the first connection part 12 or the second connection part 13 by a threaded fastener.

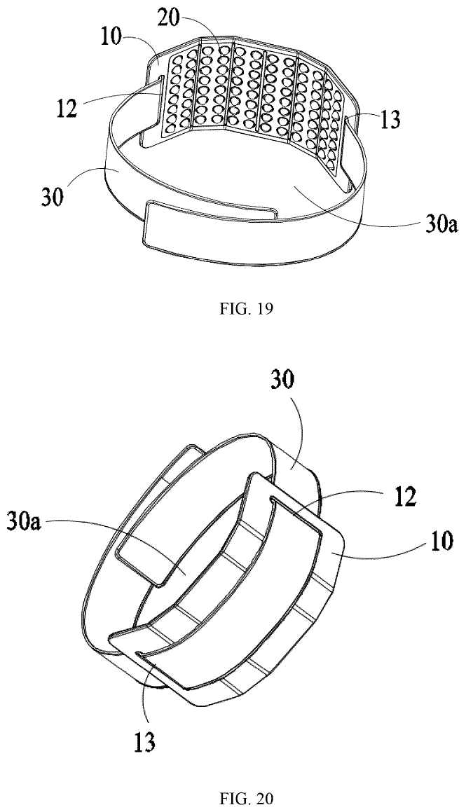

Referring to FIG. 19 and FIG. 20, when the connection structure provided between the first winding band 30 and the heat-conducting housing 10 is a thread-through structure, the first connection portion 12 and the second connection portion 13 are connection holes. When the first connection portion 12 and the second connection portion 13 are connection holes 25, the first winding band 30 passes through the first connection portion 12 and the second connection portion 13, and a front end and a terminal end of the first winding band 30 are connected to form a winding space 30a.

Referring to FIG. 8~9, the phototherapy device 100 is further provided with a second winding band 40, the second winding band 40 is provided between the second connection portion 1312 and the first winding band 30, an end of the second winding band 40 is connected to the second connection portion 13, and another end of the second winding band 40 is connected to another end of the first winding band 30, so as to facilitate the connection of another end of the first winding band 30 to the second connection portion 13 through the second winding band 40, thereby facilitating the winding space 30a to expand or reduce the size of the winding space 30a with the adjustment of the second winding band 40, further expanding the range to be threaded by the winding space 30a, such that the adaptability of the heat-conducting housing 10 is improved. Optionally, the second winding band 40 is an elastic second winding band 40.

Referring to FIGS. 10 to 16, the heat-conducting housing 10 is provided with a backside connection portion 14, the backside connection portion 14 is provided on the side of the heat-conducting housing 10 that is backward to the light-transmitting portion 11 and toward a rear side of the heat-conducting housing 10. The backside connection portion 14 is used to connect the to-be-connected member 50, so as to facilitate the connection of the backside connection portion 14 to the to-be-connected member 50.

Referring to FIGS. 10 to 16, the backside connection portion 14 is detachably connected to the to-be-connected member 50, so that the backside connection portion 14 can be connected to or detached from the to-be-connected member 50, and when the backside connection portion 14 is connected to the to-be-connected member 50, the backside connection portion 14 can be supported by the to-be-connected member 50, so as to facilitate the to-be-connected member 50 to support the heat-conducting housing 10 and the phototherapy module 20. When the backside connection portion 14 is detached from the to-be-connected member 50, so as to facilitate the heat-conducting housing 10 to be detached from the to-be-connected member 50, which improves the replacement convenience and maintenance convenience of the heat-conducting housing 10 and the phototherapy module 20, and avoids the heat-conducting housing 10 being unable to be detached from the to-be-connected member 50.

Referring to FIGS. 10 to 16, the detachable structure provided between the backside connection portion 14 and the to-be-connected member 50 is a magnetic suction structure, a snap connection structure, a threaded connection structure, a locking connection structure, and a glued connection structure, at this time, the backside connection portion 14 is detachably connected to the to-be-connected member 50 by way of magnetic suction, or, the backside connection portion 14 is detachably connected to the to-be-connected member 50 by way of magnetic suction snap-fit, or, the backside connection portion 14 is detachably connected to the to-be-connected member 50 by means of threading, or, the backside connection portion 14 is detachably connected to the to-be-connected member 50 by means of locking, or, the backside connection portion 14 is detachably connected to the to-be-connected member 50 by means of gluing.

Optionally, the backside connection portion 14 is detachably connected to the heat-conducting housing 10, and the heat-conducting housing 10 is provided with a accommodation slot and a telescopic member, which is retractably connected to the heat-conducting housing 10 and protrudes out of the accommodation slot. The accommodation slot is configured for insertion of the backside connection portion 14, and the telescopic member is inserted into the backside connection portion 14 after the backside connection portion 14 is inserted into the accommodation slot, such that the backside connection portion 14 is connected to the heat-conducting housing 10.

Optionally, the heat-conducting housing 10 is swingably connected to the backside connection portion 14, and the heat-conducting housing 10 swings in the direction of the axis at the connection between the heat-conducting housing 10 and the backside connection portion 14, so as to facilitate adjustment of the position of the heat-conducting housing 10 relative to the backside connection portion 14, thereby facilitating the phototherapy module 20 to swing along with the swing of the heat-conducting housing 10, in order to adjust an angle of the light output from the phototherapy module 20 relative to the user.

Referring to FIGS. 10 to 16, the heat-conducting housing 10 is further provided with a swinging portion 15, the swinging portion 15 is provided at a rear side of the backside connection portion 14 and swingably connected to the backside connection portion 14, so as to facilitate adjustment of the position of the swinging portion 15 relative to the backside connection portion 14, the heat-conducting housing 10 swings relative to the backside connection portion 14 as the swinging portion 15 swings relative to the backside connection portion 14, such that the heat-conducting housing 10 swings relative to the backside connection portion 14 by the swinging portion 15 to adjust the angle of the heat-conducting housing 10. Optionally, the swinging portion 15 is hinged to the backside connection portion 14, such that the swinging portion 15 can swing relative to the backside connection portion 14.

Optionally, the swinging portion 15 is detachably provided to the heat-conducting housing 10 and is quickly provided to the heat-conducting housing 10, at which time the heat-conducting housing 10 is provided with an elastic snap 16, the swinging portion 15 snaps together with the elastic snap 16 during mounting to the heat-conducting housing 10, and the elastic force of the elastic snap 16 is fully utilized to make the swinging portion 15 quickly mounted with the heat-conducting housing 10. Optionally, the elastic snap 16 performs elastic expansion and contraction in the up and down direction or swinging expansion and contraction in the forward and backward direction relative to the heat-conducting housing 10.

Referring to the accompanying FIGS. 10 to 16, the to-be-connected member is at least one of a multi-angle stand, a floor stand, a telescopic stand, and a stylized stand, and the backside connection portion 14 is connected to the to-be-connected member and is compatible with a variety of use scenarios.

When the to-be-connected member 50 is a multi-angle holder, the multi-angle holder is provided with a support seat 51, a plurality of support arms 52 and a clamping seat 53. The plurality of support arms 52 are provided between the support seat 51 and the clamping seat 53, and the plurality of support arms 52 are hinged with each other, so as to facilitate the mutual movement of the plurality of support arms 52, so as to facilitate the adjustment of the position of the plurality of support arms 52, and the plurality of support arms 52 can adjust a spatial position of the heat-conducting housing 10. The support seat 51 is connected to one support arm 52, and the clamping seat 53 is connected to the other support arm 52, so as to facilitate the support seat 51 to be movable relative to the clamping seat 53 through the plurality of support arms 52, such that the position of the support seat 51 relative to the clamping seat 53 can be adjusted.

Referring to FIGS. 10 to 16, the backside connection portion 14 is connected to the support seat 51, so that the backside connection portion 14 is connected to the support seat 51, thereby facilitating the backside connection portion 14 to be movable along with the activity of the support seat 51, to adjust a position of the backside connection portion 14. The clamping seat 53 clamps the to-be-clamped member, so as to facilitate the connection of the clamping seat 53 to the to-be-clamped member, and to ensure that the position of the clamping seat 53 is adjusted relative to a position of the to-be-clamped member, thereby facilitating the clamping seat 53 to be in a stationary state relative to the to-be-clamped member, such that the multi-angle holder is connected to the to-be-clamped member through the clamping seat 53, and when the plurality of support arms 52 are movable with each other, the clamping seat 53 doesn't move, and a position of the support seat 51 is adjusted along with the activity of the plurality of support arms 52, so as to realize the activity of the support seat 51 relative to the clamping seat 53.

In this embodiment, the support seat 51 is connected to one of the plurality of support arms 52, and a locking structure 56 is provided between the support seat 51 and one of the plurality of support arms 52, the locking structure 56 includes a locking member 561, the locking member 561 is movably connected to the support arm 52 and locked or unlocked relative to the support seat 51.

At this time, the support seat 51 is provided with a first locking portion 511, and the locking member 561 is provided with a second locking portion 5611, which locks or unlocks with the first locking portion 511 relative to each other. Optionally, both the first locking portion 511 and the second locking portion 5611 are engaged teeth.

Further, the locking structure 56 further includes an elastic member 562, the elastic member 562 is provided between the support arm 52 and the locking member 561 and drives the locking member to be locked in the support seat 51. Optionally, the elastic member 562 is a spring.

Further, the locking structure 56 further includes a key 563, the key 563 is movably provided on the support arm 52 and connected to the locking member 561, the locking member 561 is detached from the support arm 52 as the key 563 is pressed by human and the unlocking of the locking member 561 from the support arm 52 is realized, thereby realizing a free state of the support seat 51 and the support arm 52 for adjusting the position of the support seat 51 relative to the support arm 52.

When the user's hand is detached from the key 563, the key 563 is non-pressed state, and the elastic member 562 exerts an elastic force on the locking member 561 and drives the movement of the locking member 561 toward the support seat 51, thereby realizing the locking of the locking member 561 and the support arm 52, thereby realizing the locking state of the support seat 51 and the support arm 52, in order to facilitate the maintenance of the position of the support seat 51 relative to the support arm 52. When the to-be-connected member 50 is a floor stand, the multi-angle holder is provided with a floor stand 54 and a support frame 55, the floor stand 54 is arranged on the ground, the support frame 55 is connected to the floor stand 54 and supports a plurality of support arms 52 and the support seat 51, the plurality of support arms 52 are located between the support frame 55 and the support seat 51, and the plurality of support arms 52 can adjust the spatial position of the heat-conducting housing 10, at this time, the plurality of support arms 52 are are articulated with each other, so as to facilitate the plurality of support arms 52 to be movable with each other, such that the support seat 51 can move relative to the support frame 55 through the plurality of support arms 52, thereby facilitating the adjustment of the position of the support seat 51 relative to the support frame 55. Optionally, the floor seat 54 and the support frame 55 replace the clamping seat 53.

Further, the support frame 55 is telescopically connected to the floor seat 54, and the height position of the support frame 55 is realized by the telescopic structure between the support frame 55 and the floor seat 54, thereby adjusting the spatial position of the heat-conducting housing 10, and adjusting the height position of the heat-conducting housing 10.

In the above embodiments, the description of each embodiment has its own focus, and the parts that are not detailed in a certain embodiment can be seen in the relevant description of other embodiments.

In the description of the present application, the terms “first” and “second” are used for descriptive purposes only, and are not to be understood as indicating or implying relative importance or implicitly specifying the number of technical features indicated. As a result, a feature defined with “first” or “second” may include one or more features, either explicitly or implicitly.

Herein adopts specific examples for elaboration of the principle and implementation of the present application, the illustration of the above examples are only used to help understand the method and the core ideas of the present application; and the those skilled in the art, based on the idea of the present application, will make some change in the specific implementation of the application and the scope of the application, in summary, the contents of this specification should not be construed as a limitation on the present application.

What is claimed is:

1. A phototherapy device, comprising:
a heat-conducting housing, capable of curved surface deformation under an external force; wherein the heat-conducting housing has a curved surface state or a planar state, and the heat-conducting housing is further provided with a light-transmitting portion;
a phototherapy module, provided in the heat-conducting housing;
wherein the phototherapy module is provided with a light output portion, and the light output portion is arranged corresponding to the light-transmitting portion for outputting light, which penetrates the light-transmitting portion and is emitted outwardly; and
the phototherapy module exchanges heat with the heat-conducting housing, and the phototherapy module generates heat when outputting light, and the heat is dissipated outwardly via the heat-conducting housing.

2. The phototherapy device according to claim 1, wherein the heat-conducting housing is provided with an accommodation slot, the accommodation slot is arranged in correspondence with the light-transmitting portion; and
the phototherapy module is accommodated in the accommodation slot, the phototherapy module is in direct contact with an inner side wall of the accommodation slot and exchanges heat with the heat-conducting housing.

3. The phototherapy device according to claim 2, wherein the heat-conducting housing is a heat-conducting member, the heat-conducting housing is exposed to an external environment and dissipates heat generated by the phototherapy module outwardly in different directions.

4. The phototherapy device according to claim 3, wherein the heat-conducting housing is a silicone housing, a silicone plate, a heat-conducting metal plate or a heat-conducting sheet.

5. The phototherapy device according to claim 2, wherein the phototherapy module comprises a circuit board and a plurality of lamp beads;
the circuit board is connected to an inner side wall of the accommodation slot, and the circuit board is in direct contact with the inner side wall of the accommodation slot; and
a plurality of the lamp beads are provided on a side of the circuit board facing the light-transmitting portion, and are electrically connected to the circuit board and output light to the light-transmitting portion and to the external environment, to perform a phototherapy action by the light.

6. The phototherapy device according to claim 5, wherein a plurality of the phototherapy modules are provided and spaced apart along a lengthwise direction of the heat-conducting housing and extended along a widthwise direction of the heat-conducting housing.

7. The phototherapy device according to claim 6, wherein articulation members are provided among the plurality of the phototherapy modules, and two adjacent phototherapy modules are swung through the articulation members for angle adjustment.

8. The phototherapy device according to claim 1, wherein the heat-conducting housing is provided with a first connection portion and a second connection portion;
the phototherapy device further comprises a first winding band, and the first winding band is connected to the heat-conducting housing;
one end of the first winding band is connected to the first connection portion and another end of the first winding band is directly or indirectly connected to the second connection portion; and
the first winding band is provided with a winding space, and the winding space is configured for a body of a user to pass through.

9. The phototherapy device according to claim 1, wherein the heat-conducting housing is provided with a backside connection portion, and the backside connection portion is configured for connecting a to-be-connected member;
the backside connection portion is detachably connected to the to-be-connected member and is supported by the to-be-connected member;
a detachable structure provided between the backside connection portion and the to-be-connected member is a magnetic suction structure, a snap connection structure, a threaded connection structure, a locking connection structure, and a glued connection structure; and
the to-be-connected member is at least one of a multi-angle holder, a floor holder, a telescopic holder, and a stylized holder.

10. The phototherapy device according to claim 9, wherein the heat-conducting housing is swingably connected to the backside connection portion; and
the heat-conducting housing is further provided with a swinging portion, the swinging portion is provided on one side of the backside connection portion and swingably connected to the backside connection portion, and the heat-conducting housing swings relative to the backside connection portion as the swinging portion swings relative to the backside connection portion.

11. The phototherapy device according to claim 10, wherein the heat-conducting housing is provided with an elastic snap, the swinging portion snaps with the elastic snap during installation on the heat-conducting housing, and an elastic force of the elastic snap is configured for installation of the swinging portion and the heat-conducting housing; and
the elastic snap is elastically telescoped in an up and down direction or oscillated telescoped in a forward and backward direction relative to the heat-conducting housing.

12. The phototherapy device according to claim 1, wherein the heat-conducting housing serves as a support component of the phototherapy device, and the heat-conducting housing is capable of curved surface deformation under the external force, to undergo multiple shape changes.

13. The phototherapy device according to claim 1, wherein the heat-conducting housing has a curved state or a planar state to facilitate heat dissipation in different forms.

14. The phototherapy device according to claim 13, wherein when the heat-conducting housing is in the planar state to adapt to a planar phototherapy module, and the phototherapy module is in the planar state to output light to carry out the planar phototherapy on the user; and
when the heat-conducting housing is in the curved state, the heat-conducting housing is adapted to the phototherapy module of curved surface, an outer surface of the heat-conducting housing is in curved attachment with a waist or arm of the user.

15. The phototherapy device according to claim 8, wherein the first winding band is capable of elastic stretching under the external force, and the first winding band elastically regulates the winding space to adjust a size of the winding space.

16. The phototherapy device according to claim 8, wherein the connection structure between the first winding band and the heat-conducting housing is at least one of a snap connection structure, a locking connection structure, a velcro connection structure, a glued connection structure, and a thread-through structure.

17. A phototherapy device, comprising:
a heat-conducting housing, being a heat-conducting member, wherein the heat-conducting housing is further provided with a light-transmitting portion;
a phototherapy module, provided in the heat-conducting housing;
wherein the phototherapy module is provided with a light output portion, and the light output portion is arranged corresponding to the light-transmitting portion for outputting light, which penetrates the light-transmitting portion and is emitted outwardly; and
the phototherapy module exchanges heat with the heat-conducting housing, and the phototherapy module generates heat when outputting light, and the heat is dissipated outwardly via the heat-conducting housing.

18. The phototherapy device according to claim 17, wherein the heat-conducting housing is provided with a backside connection portion, and the backside connection portion is configured for connecting a to-be-connected member;
the backside connection portion is detachably connected to the to-be-connected member and is supported by the to-be-connected member;
a detachable structure provided between the backside connection portion and the to-be-connected member is a magnetic suction structure, a snap connection structure, a threaded connection structure, a locking connection structure, and a glued connection structure; and
the to-be-connected member is at least one of a multi-angle holder, a floor holder, a telescopic holder, and a stylized holder.

19. The phototherapy device according to claim 17, wherein the heat-conducting housing is used as a support component of the phototherapy device, and the heat-conducting housing is capable of curved surface deformation under an external force, to carry out multiple shape changes.

20. The phototherapy device according to claim 17, wherein the heat-conducting housing has a curved state or a planar state to facilitate heat dissipation in different forms;
when the heat-conducting housing is in the planar state to adapt to a planar phototherapy module, and the phototherapy module is in the planar state to output light to carry out the planar phototherapy on a user; and
when the heat-conducting housing is in the curved state, the heat-conducting housing is adapted to the phototherapy module of curved surface, an outer surface of the heat-conducting housing is in curved attachment with a waist or arm of the user.
